(19) United States
(12) Patent Application Publication
(10) Pub. No.: US 2026/0183385 A1
(43) Pub. Date: Jul. 2, 2026

(54) PAN-COVID-19 VACCINE COMPOSITION INCLUDING CONSENSUS SEQUENCE OF SARS-COV-2 VARIANTS

(71) Applicant: KOREA NATIONAL INSTITUTE OF HEALTH, Cheongju-si, Chungcheongbuk-do (KR)

(72) Inventors: Hyun Ju IN, Cheongju-si (KR); Eun Bee CHOI, Cheongju-si (KR); Yoo Jin NA, Cheongju-si (KR); Yoo-kyoung LEE, Cheongju-si (KR)

(73) Assignee: KOREA NATIONAL INSTITUTE OF HEALTH, Cheongju-si, Chungcheongbuk-do (KR)

(21) Appl. No.: 19/428,594

(22) Filed: Dec. 22, 2025

(30) Foreign Application Priority Data
Dec. 27, 2024 (KR) 10-2024-0198555

(51) Int. Cl. A61K 39/215; A61K 39/00; A61P 31/14; C07K 14/005; C12N 7/00

(52) U.S. Cl. CPC A61K 39/215; A61P 31/14; C07K 14/005; C12N 7/00; A61K 2039/54; A61K 2039/55505; A61K 2039/575; C12N 2770/20022; C12N 2770/20034

(57) ABSTRACT

Provided are a vaccine composition for pan-COVID-19 comprising a consensus sequence of severe acute respiratory syndrome coronavirus type 2 (SARS-CoV-2) variants.

### REFERENCE TO AN ELECTRONIC SEQUENCE LISTING

The instant application contains a Sequence Listing which has been submitted electronically in XML format and is hereby incorporated by reference in its entirety. Said XML copy, created on Dec. 18, 2025, is named SequenceListing.xml and is 6,805 bytes in size.

### BACKGROUND

### Technical Field

The present invention relates to Pan-COVID-19 vaccine composition including consensus sequence of SARS-CoV-2 variants.

### Background Art

Coronavirus disease 2019 (COVID-19) caused a pandemic from March 2020 to May 2023. Despite aggressive COVID-19 vaccine development, the ongoing severe acute respiratory syndrome coronavirus 2 (SARS-CoV-2) is rapidly mutating, and the new variant weakens the effectiveness of early vaccines approved by the U.S. Food and Drug Administration (FDA), such as Moderna (mRNA-1273) and Pfizer-BioNTech (BNT162b2). Although several experiments have been conducted to design multivalent vaccine candidates, the development of a universal vaccine to protect against evolving SARS-CoV-2 variants and seasonal coronaviruses is needed.

The pan-coronavirus vaccines were designed and tested by several research groups in the following ways, nanoparticle (NP) delivery, mosaic antigens, computational antigen, consensus sequence design, and so on. For example, the 10-valent mRNA-LNP vaccine (FLUCOV-10) was designed through phylogenetic analysis using sequences from various influenza viruses and SARS-CoV-2 variants (Wuhan, BQ.1.1, BA.2.75.2, XBB1.5), and was effective in stimulating immune responses and protecting against both influenza and COVID-19. Another vaccine was a mosaic NP vaccine composed of the receptor binding domain (RBD) of omicrons (BA.1, BA.2, BA.5, BA.2.75), Delta and D614G tagged with highly conserved T cell epitope sequences from the nucleocapsid (N) or Spike (S) proteins of sarbecovirus. Mosaic NP was more potent than cocktail NP and showed broad cross-protection against multiple SARS-CoV-2 sublineages. Another group designed a pan SARS-CoV-2 vaccine using a truncated RBD from the highly conserved Omicron BA.1 variant or inserting the RBD from Delta, BA.5, or WT. Among them, delta-RBD was strongly effective in inducing neutralizing antibodies against alpha, beta, gamma, delta, Omicron subvariants (BA.1, BA.2, BA.2.75, BA.4.6, BA.5), and WT, and in preventing replication and infection of Omicron.

Preserving the SARS-CoV-2 surface glycoprotein, Spike (S), in a metastable prefusion state and the RBD in its native position is a key strategy to enhance vaccine candidate-induced immunogenicity. The first generation design spike for prefusion stabilization is S-2P (986 and 987 replaced with proline (P)). S-2P has been included in several potent vaccines, such as mRNA-1273 and BNT162b2, but has been observed to have low yields and poor thermostability. The second generation is HexaPro with Spike's four additional proline substitution at 817, 892, 899, and 942. HexaPro partially overcomes the limitations of S-2P with regard to yield and thermostability, but readily transforms the spike protein into a post-fusion state by receptor, antibody, and protease digestion. S-R/x2 is another element that maintains the spike protein in the prefusion trimer. The formation of a disulfide bond between residues 413 and 987 stabilizes the trimeric spike protein in a closed conformation.

### BRIEF DESCRIPTION OF THE INVENTION

### Technical Problem

The purpose of the present invention is to provide a vaccine composition for Pan-COVID-19 based on the consensus sequence of SARS-CoV-2 variants to protect against persistent SARS-CoV-2 mutations and seasonal coronaviruses. Therefore, in the present invention, using the open sequence, three universal recombinant vaccine candidates for COVID-19 were designed by adding factors that maintain the spike in a prefusion state to the consensus sequence of SARS-CoV-2 alpha, beta, gamma, delta, and omicron subvariants (BA.1, BA.2, BA.2.12.1, BA.2.75, BA.4, BA.5).

### Technical Solution

In order to achieve the purpose of the present invention, the present invention provides a recombinant protein derived from the spike protein of severe acute respiratory syndrome coronavirus 2 (SARS-CoV-2) having SEQ ID NO: 1, a recombinant protein derived from the spike protein of severe acute respiratory syndrome coronavirus 2 (SARS-CoV-2) having SEQ ID NO: 2, or a recombinant protein derived from the spike protein of severe acute respiratory syndrome coronavirus 2 (SARS-CoV-2) having SEQ ID NO: 3.

### Effects of the Invention

The present invention relates to a pan-COVID-19 vaccine composition, wherein a recombinant protein based on the consensus sequence of SARS-CoV-2 alpha, beta, gamma, delta and omicron subvariants is proposed as a vaccine for protecting against persistent SARS-CoV-2 mutations and seasonal coronaviruses.

### BRIEF DESCRIPTION OF THE DRAWINGS

FIG. 1 illustrates the design of a recombinant vaccine candidate for pan-COVID-19. A) Pie and bar charts are shown for the number of variants used in sequence analysis and the mutation rate derived from multiple sequence alignment (MSA), respectively. B) Schematic diagram of three recombinant vaccine constructs including several factors (S-GSAS/6P, S-R/x2, etc.) known to enhance stability and mutation through MSA analysis.

FIG. 2 shows the humoral immune response of a recombinant vaccine candidate in C57BL/6 mice. A) Immunization schedule for binding and neutralizing antibody measurements. C57BL/6 mice were immunized with the candidate or alum via intramuscular injection as scheduled. B) One week after the second vaccination, pooled serum samples (n=5/group) from mice immunized with alum or vaccine candidates were used to confirm neutralizing antibodies against each variant by the plaque reduction neutralization test (PRNT) method. C) SARS-CoV-2 S protein-specific IgG was detected by enzyme-linked immunosorbent assay (ELISA) using serially diluted sera from C57BL/6 mice. The cutoff value was calculated using the mathematical formula “mean optical density (OD) of the negative control+3 times the standard deviation of the negative control” to determine the final titer in ELISA without a positive standard.

FIG. 3 shows the humoral immune response of the recombinant vaccine candidate in C57BL/6 mice. A) Immunization schedule. C57BL/6 mice were immunized with the candidate or alum via intramuscular injection as scheduled. B) One week after the third vaccination, neutralizing antibodies against each variant were identified using the PRNT method using pooled serum samples (n=5/group) from mice immunized with alum or vaccine candidates. C) SARS-CoV-2 S protein-specific IgG was detected by ELISA using serially diluted sera from C57BL/6 mice.

FIG. 4 shows the cellular immune response of the recombinant vaccine candidate in C57BL/6 mice. A) Immunization schedule to measure T cell responses. B) Using an enzyme-linked immunosorbent spot (ELISpot) plate reader, the amount of interferon (IFN) gamma (γ) secreted from immunized mouse splenocytes one week after the third vaccination was analyzed using staining intensity. C) IFN-γ ELISPOT image. D) IL-2 was measured using a mouse Quantikine ELISA kit. Splenocyte culture supernatant from immunized mice was used as a sample. Data are presented as means with standard errors and were evaluated using an unpaired t-test. A P value less than 0.05 was considered significant.

FIG. 5 shows the protective efficacy of a recombinant vaccine candidate in K18-hACE2 transgenic mice. A) K18-hACE2 mice were vaccinated with candidate substances or alum via intramuscular injection as scheduled. Then, 4 weeks after the boost, Wuhan (2.6×10⁵ PFU) and XBB1.5 (2.9×10⁵ PFU) were intranasally inoculated. B) To visualize the protection against infection following vaccination, a red-dye-labeled RNAscope was used to amplify hybridization signals with the target sequence (SARS-CoV-2 Spike) on mouse lung tissue. In situ hybridization (ISH) scores were calculated from the stained areas on days 3 and 5 post-infection. Score 0: No lesion, 1: 2-20%, 2: 21-50%, 3: 51-80%, and 4: 81-100%. C) Representative 10× images. Data are presented as means with standard errors and were evaluated using an independent-samples t-test. A P value less than 0.05 was considered significant.

### DETAILED DESCRIPTION OF THE INVENTION

The term “protein” as used herein is generally defined as a chain of amino acid residues having a defined sequence. The term also comprises modified amino acid polymers.

In one example of the present invention, the present invention provides a vaccine composition comprising the recombinant protein.

The vaccine composition may further comprise one or more selected from the group consisting of preservatives, diluents, adjuvants and carriers, and the adjuvants may be alum.

Suitable carriers for vaccines are known to those skilled in the art and include, but are not limited to, proteins, sugars, etc. The carriers may be an aqueous solution, or a non-aqueous solution, a suspension, or an emulsion. Structured or amorphous organic or inorganic polymers may be used as immunoadjuvants to increase immunogenicity.

Immunoadjuvants are generally known to play a role in promoting immune responses through chemical and physical binding to antigens. As immunoadjuvants, amorphous aluminum gel, oil emulsion, or double oil emulsion, and immunosol, etc. can be used.

Additionally, various plant-derived saponins, levamisole, CpG dinucleotide, RNA, DNA, LPS, and various types of cytokines, etc. can be used to stimulate the immune response. The immune composition can be used to induce an optimal immune response by combining various adjuvants and immune-stimulating additives. Furthermore, stabilizers, inactivators, antibiotics, preservatives, and other additives can be added to the vaccine. Depending on the route of administration, the vaccine antigen may also be mixed with distilled water, buffer solutions, and other solutions.

The vaccine composition can be formulated and used in the form of oral formulations such as powders, granules, tablets, capsules, suspensions, emulsions, syrups, aerosols, and in the form of topical preparations, suppositories, or unit dose ampoules or multiple dose injections, each according to a conventional method. When formulating the vaccine composition, it can be prepared by adding a diluent or excipient such as a commonly used filler, bulking agent, binder, wetting agent, disintegrant, or surfactant.

When the vaccine composition is prepared as a parenteral formulation, it can be formulated into the form of an injection, a transdermal administration agent, a nasal inhalant, and a suppository according to a method known in the art together with a suitable carrier. When formulated as an injection, suitable carriers include sterile water, ethanol, polyols such as glycerol or propylene glycol, or mixtures thereof, and preferably, Ringer's solution, phosphate buffered saline (PBS) containing triethanolamine, sterile water for injection, or an isotonic solution such as 5% dextrose. When formulated as a transdermal agent, it can be formulated in the form of ointment, cream, lotion, gel, external solution, paste, liniment, or aerosol. In the case of nasal inhalation, it can be formulated in the form of an aerosol spray using a suitable propellant such as dichlorofluoromethane, trichlorofluoromethane, dichlorotetrafluoroethane, or carbon dioxide, and in the case of formulating it as a suppository, the base can be witepsol, tween 61, polyethylene glycol, cacao butter, laurin butter, polyoxyethylene sorbitan fatty acid ester, polyoxyethylene stearate, sorbitan fatty acid ester, etc.

The route of administration of the vaccine composition may be through any general route as long as it can reach the target tissue, and specifically, the vaccine composition may be selected from the group consisting of compositions for intramuscular administration, subcutaneous administration, intraperitoneal administration, intravenous administration, oral administration, dermal administration, ocular administration, nasal administration, and intracerebral administration.

The vaccine composition can be administered in a pharmaceutically effective amount, wherein the term “pharmaceutically effective amount” means an amount sufficient to treat or prevent a disease at a reasonable benefit/risk ratio applicable to medical treatment or prevention, and the effective dosage level can be determined according to factors including the severity of the disease, the activity of the drug, the patient's age, weight, health, sex, the patient's sensitivity to the drug, the time of administration of the composition of the present invention used, the route of administration and the excretion rate, the treatment period, the drug used in combination with or concurrently with the composition of the present invention used, and other factors well known in the medical field. The vaccine composition can be administered alone or in combination with ingredients known to exhibit known preventive or therapeutic effects. It is important to consider all of the factors and administer the amount that achieves maximum efficacy with the minimum amount possible without adverse effects.

The dosage of the vaccine composition can be determined by a person skilled in the art in consideration of the purpose of use, the degree of toxicity of the disease, the patient's age, weight, sex, medical history, or the type of substance used as the active ingredient. For example, the vaccine composition of the present invention can be administered to an adult at about 0.1 ng to about 1,000 mg/kg, preferably 1 ng to about 100 mg/kg, and the frequency of administration of the composition of the present invention is not particularly limited thereto, but can be administered once a day or administered several times in divided doses. The dosage or frequency of administration does not limit the scope of the present invention in any way.

The vaccine composition is for the prevention of severe acute respiratory syndrome coronavirus 2 (SARS-CoV-2) infection, wherein severe acute respiratory syndrome coronavirus 2 (SARS-CoV-2) infection disease is coronavirus disease-19 (COVID-19), and the severe acute respiratory syndrome coronavirus 2 (SARS-CoV-2) is at least one selected from the group consisting of alpha, beta, gamma, delta and omicron, more preferably alpha (B.1.1.7), beta (B.1.351), gamma (P.1), delta (B.1.617.2) and omicron (BA.1, BA.2, BA.2.12.1, BA.2.75, BA.4, BA.5), but is not limited thereto.

The vaccine composition may be used as one or more selected from the group consisting of a priming vaccine composition and a boosting vaccine composition, and preferably, the boosting vaccine composition may be administered twice or more, but is not limited thereto.

In another example of the present invention, the present invention provides a method for preventing severe acute respiratory syndrome coronavirus 2 (SARS-CoV-2) infection in a non-human mammal, comprising administering the vaccine composition. The administering step may include a step of inducing a priming immune response; and a step of inducing a boosting immune response.

Hereinafter, preferred embodiments of the present invention will be described in detail. Furthermore, the following description includes numerous specific details, such as specific components. These details are provided solely to facilitate a more comprehensive understanding of the present invention. It will be apparent to those skilled in the art that the present invention can be practiced without these specific details. Further, when describing the present invention, if it is determined that a detailed description of a related known function or configuration may unnecessarily obscure the gist of the present invention, the detailed description is omitted.

### EXAMPLES

### Example 1. Design of a Recombinant Protein Vaccine

894,562 SARS-CoV-2 sequences were downloaded from the Global Initiative for Sharing All Influenza Data (GISAID). The collected variants of concern (VOC) sequences, namely alpha (B.1.1.7), beta (B.1.351), gamma (P.1), delta (B.1.617.2), and omicron sub-variants (BA.1, BA.2, BA.2.12.1, BA.2.75, BA.4, BA.5), were aligned with the reference sequence (hCoV-19/Wuhan/WIV04/2019), and point mutations and insertion-deletion mutations (Indels) were detected using the nextalign tool. Then, multiple sequence alignment (MSA) analysis was performed using MAFT tool to obtain consensus sequence (cut-off, 0.6). To design a stable universal recombinant vaccine candidate for COVID-19, A570D, T7161, S982A, and D1118H were included in the consensus sequence, and S-GSAS/6P and S-R/x2 were added to induce an effective immune response. To enhance protein secretion and expression and to form a trimeric structure of the spike protein, a signal peptide at the N-terminus and a foldon (Fd) sequence at the C-terminus were also added, respectively.

### Example 2. Viruses and Cell Lines

Wild-type (Wuhan, NCCP43326), alpha (GRY lineage, B.1.1.7, NCCP4381), beta (GH lineage, B.1.351, NCCP4382), gamma (GR lineage, P.1, NCCP43388), delta (GK lineage, AY.69, NCCP43409), and omicron subvariants (GRA lineage, BA.1, NCCP43408; BA.1.1, NCCP43411; BA.2, NCCP43412; XBB.1.5, NCCP43440) SARS-CoV-2 viruses were provided by the National Culture Collection for Pathogens (NCCP) and titered by plaque assay. All infections with live virus within cells were performed in a biosafety level 3 (BL3) facility. Vero E6 (African green monkey kidney cells) were purchased from ATCC (American type culture collection, USA) and cultured in DMEM (Gibco, USA) medium supplemented with 10% fetal bovine serum (FBS) and L-glutamine in a humidified incubator at 37° C. with 5% CO₂.

### Example 3. Animal Immunization

The vaccine candidate was injected intramuscularly with alum every three weeks into five C57BL/6 mice (SAMTACO Bio Korea; Gyeonggi-do, Korea) per group, aged 5 to 7 weeks. Mouse serum was collected one week after each boosting, and mouse spleens were extracted one week after the final vaccination to determine humoral and cellular immune responses. Mouse samples from the same group were pooled and used in the experiments.

For each group, 10 K18-hACE2 mice (Jackson Laboratory, USA), aged 5 to 7 weeks, were injected intramuscularly twice at 3-week intervals with the vaccine candidate mixed with alum. At week 7, Wuhan (2.6×10⁵ PFU) and XBB1.5 (2.9×10⁵ PFU) were injected intranasally into mice to evaluate the protective effect. Autopsies were performed 3 and 5 days after viral infection to measure virus in lung tissue. All experimental procedures were approved by the Institute Animal Care and Use Committee (IACUC) of the Korea Disease Control and Prevention Agency (KDCA-IACUC-22-032) in accordance with the guidelines for laboratory animal ethics.

### Example 4. Plaque Reduction Neutralization Test (PRNT)

Per well, 2.5×10⁵ Vero E6 cells were added with a mixture of serially diluted serum and virus (50 Plaque-forming unit, PFU) mixed one hour before and incubated for 1 hour. Cells cultured in 2×MEM overlay medium (1.5% agarose (Lonza, USA)) containing 4% FBS (Gibco, USA) for 3 days after infection were stained with crystal violet. The plaques in each well were counted and the neutralizing titer (NT₅₀) was calculated using the Karber formula.

### Example 5. Enzyme-Linked Immunosorbent Assay (ELISA)

To measure SARS-CoV-2 S-specific IgG antibodies in immunized serum samples, each well of a 96-well plate was coated with 50 ng SARS-CoV-2 Spike S1+S2 ECD-His recombinant protein (Sino Biological). After blocking the antigen-coated plate with 1% bovine serum albumin (BSA), serially diluted mouse serum samples were added and reacted at 37° C. for 1 hour. Then, goat anti-mouse IgG-HRP (Santa Cruz Biotechnology, 1:5000) was used as the secondary antibody. Tetramethyl benzidine (TMB) substrate (Invitrogen, USA) and stop solution (GenDEPot) were added to measure the optical density at 450 nm. Washing with PBST (0.02% Tween-20 in PBS) was included between each step.

To determine cytokine expression, Quantikine sandwich ELISA (R&D Systems, USA) was performed according to the manufacturer's instructions. The optical density of IL-2 was measured using a microplate reader set to 450 nm.

### Example 6. ELISpot Analysis

To evaluate the cellular immune response, the amount of IFN-γ secretion was observed using a mouse IFN-γ ELISpot kit (R&D Systems, USA). Splenocytes from immunized mice were filtered through a 50 μm pore size nylon cell strainer (BD, USA) and erythrocytes were removed with ACK buffer solution. 1×10⁶ isolated splenocytes were plated on 96-well polyvinylidene difluoride (PVDF) membrane plates coated with mouse IFN-γ-specific monoclonal antibodies and then stimulated with 1 μg/ml PepMix SARS-CoV-2 Spike variants (JPT, Germany) for 24 hours. The next day, cells were removed and IFN-γ secreted by stimulated cells was detected with biotinylated IFN-γ mouse antibody. The plates were then incubated with alkaline phosphatase conjugated streptavidin and BCIP/NBT substrate was added to monitor colored spot formation. Washing steps were included between each process, and the color spot intensity was measured with an Immunospot reader (C.T.L., USA).

### Example 7. In Situ RNA Detection

To visualize the protective effect of vaccination, lung tissues were obtained 3 and 5 days after infection with SARS-CoV-2 variants (wild type, XBB1.5). SARS-CoV-2 infection status in formalin-fixed paraffin-embedded (FFPE) tissues was detected by RNAscope probes. The in situ hybridization scores based on labeling amplification are as follows: score 0 indicates no labeling, score 1 indicates up to 20% positivity in the tissue, score 2 indicates 21-50% positivity, score 3 indicates 51-80% positivity, and score 4 indicates 81-100% positivity. A scoring system was introduced to determine the extent of lung lobe infection.

### Experimental Example 1. Design of the Universal Recombinant Vaccines with a Consensus Spike Protein Sequence

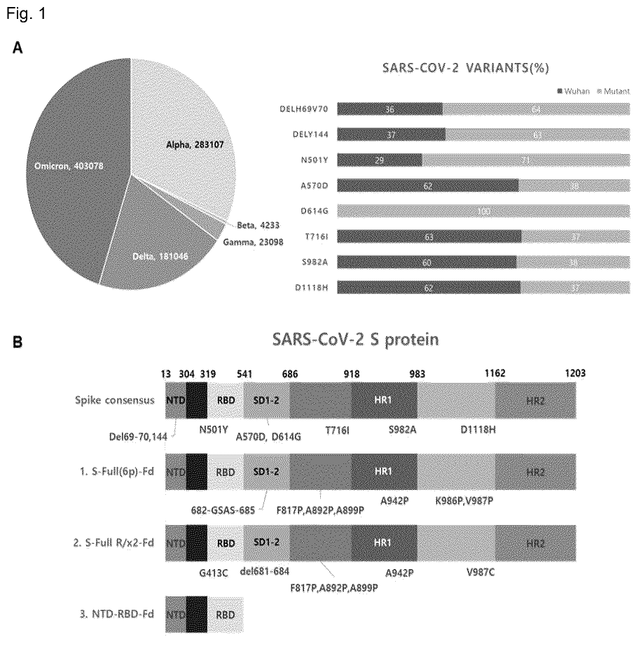

To construct a universal recombinant vaccine, 894,562 spike protein amino acid sequences were downloaded from the GISAID database. The number of each variant used in the analysis is shown in FIG. 1A and the respective proportions are as follows: alpha 31.6%, beta 0.5%, delta 20.2%, gamma 2.6%, and omicron 45.1%. D614G, N501Y, DELH69V70, and DELY144 are consensus sequences that show more than 60% identity among the entire sequences in multiple sequence analysis (MSA). Additionally, A570D, T7161, S982A, and D1118H (alpha mutations) were added to the analyzed spike consensus sequence to improve the stability of the vaccine candidate, and S-GSAS/6P and S-R/x2 were added to increase immunogenicity. In the present invention, three universal vaccine candidates for COVID-19 were designed based on computational analysis (FIG. 1B).

### Experimental Example 2. The Universal Vaccine Candidates Elicit a Humoral Immune Response Against SARS-CoV-2 Variants

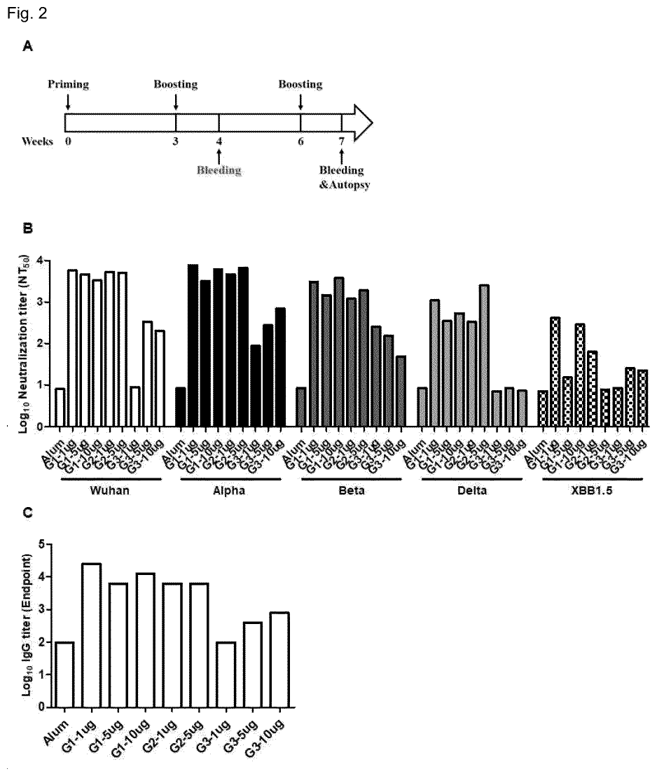

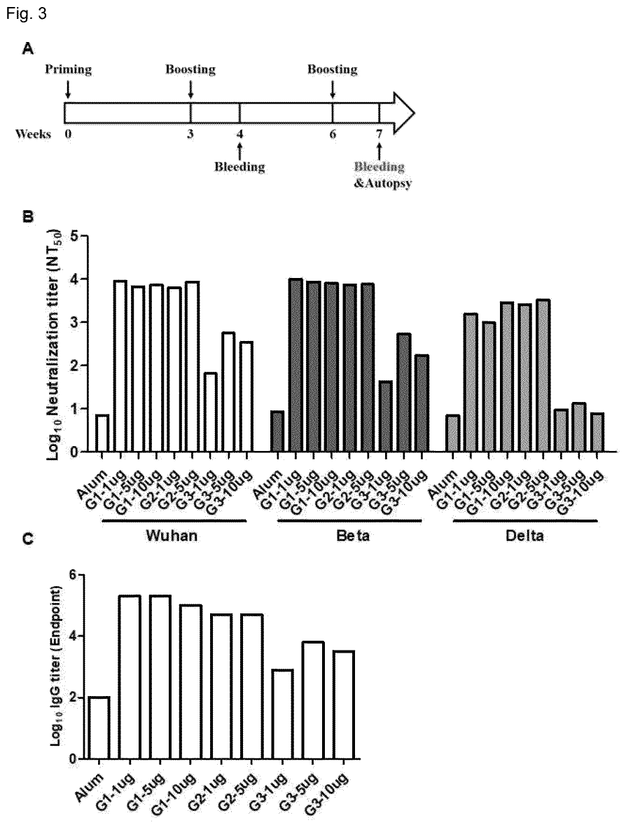

The immunization schedule to determine humoral immunity of the universal vaccine candidates is shown in FIG. 2A. Five C57BL/6 mice per group were injected intramuscularly with 1, 5, or 10 μg of the candidate compound at 3-week intervals. Mouse serum was collected 1 week after the first and second boost vaccinations (red). All data in FIG. 2 were obtained from pooled mouse serum collected one week after the first boost. To evaluate neutralizing antibodies against various variant SARS-CoV-2, PRNT was performed using wild-type, alpha, beta, delta, and XBB1.5 SARS-CoV-2 (FIG. 2B). The designed universal vaccine candidates induced neutralization titers (NT) across all variants, but candidates 1 and 2 were more potent than candidate 3. In contrast, there was no delta neutralizing activity for candidate 3. SARS-CoV-2 spike-specific total IgG was also increased by all candidates, particularly candidates 1 and 2 (FIG. 2C). Humoral immune responses after the second boost were also examined in FIG. 3A-C. The second boost showed a slightly higher immune induction capacity compared to the first boost vaccination, but was similar. For convenience, the candidate is denoted as G (candidate 1=G1).

### Experimental Example 3. Cellular Immune Response Induced by the SARS-CoV-2 Universal Vaccine Candidates

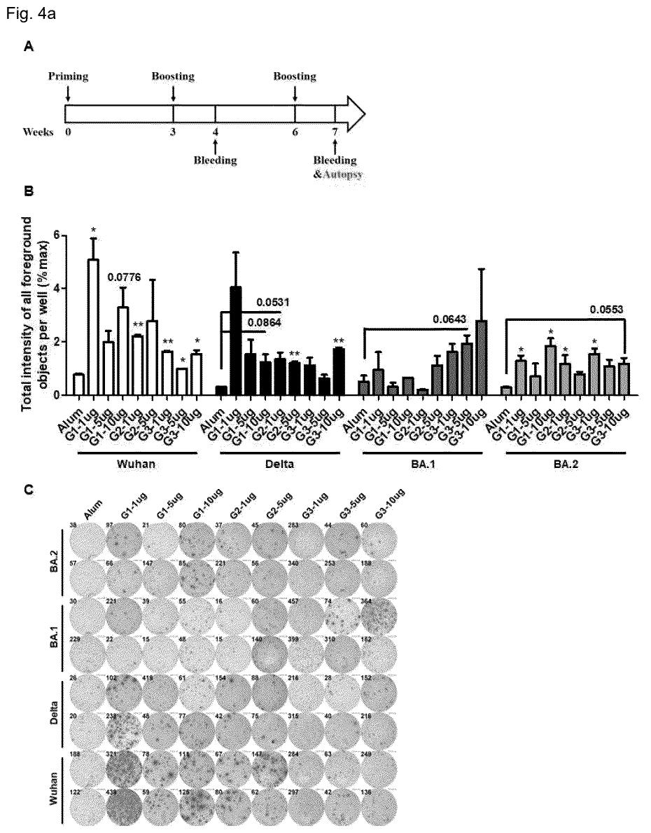

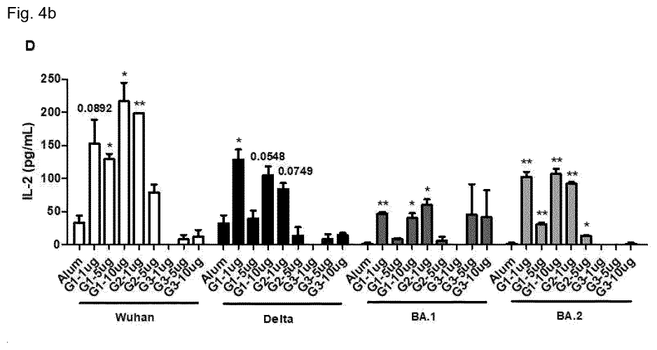

FIG. 4A shows the immunization schedule to investigate the cellular immune response induced by a second boost of the universal vaccines (red). We examined the amount of IFN-γ secretion using immunized mouse splenocytes stimulated with each SARS-CoV-2 peptide pool, such as wild-type, delta, BA.1, and BA.2, and confirmed IFN-γ induction, although the degree varied between the candidates (FIG. 4B-C). Another way to determine antigen-specific T cell responses was to measure cytokine levels secreted by immunized mouse splenocytes. Interleukin 2 (IL-2) levels were strongly induced by G1/2 rather than G3 in almost all mutants (FIG. 4D).

### Experimental Example 4. Protective Efficacy of the Universal Vaccine Candidates Against SARS-CoV-2 Challenge

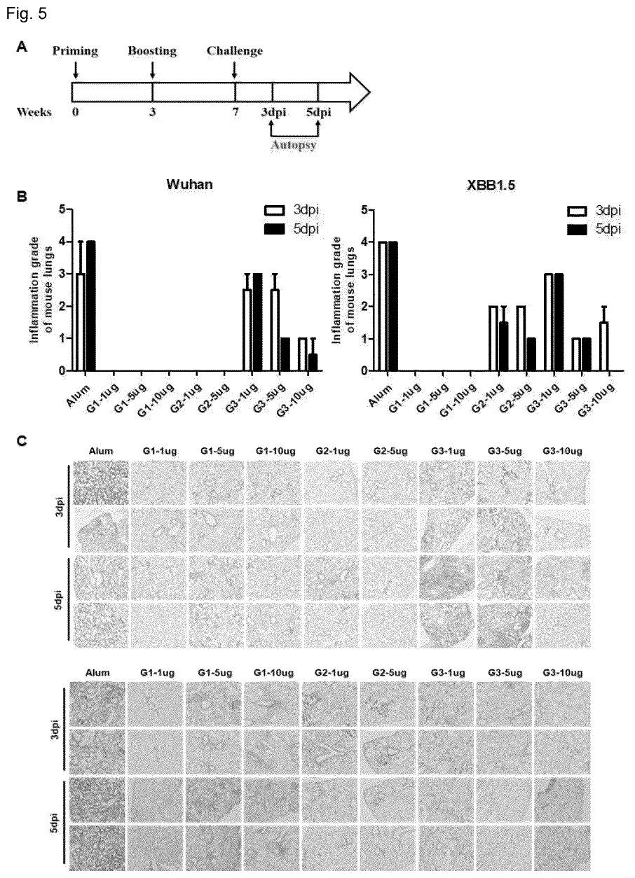

To further investigate the protective efficacy of the candidates, K18-hACE2 transgenic mice were immunized according to the schedule shown in FIG. 5A and then infected with wild-type and XBB1.5 SARS-CoV-2. The infection protection ability of the candidates was confirmed using a SARS-CoV-2 RNA probe (target: spike, nucleotide position: 21631-23303 base pairs, number of pairs: 20) that can detect not only the wild type but also various mutant viruses such as alpha, beta, gamma, and XBB1.5 in FIGS. 5B and 5C. In situ hybridization scores of mouse lung tissues were observed to be lower in G1 and G2 compared to G3 in both wild-type and XBB1.5.

[Sequence Listing]

Candidate 1 (SEQ ID NO: 1): U4579IC070-1_S full(6P)-Fd
MGWSCIILFLVATATGVHSQCVNLTTRTQLPPAYTNSFTRGVYYPDKVFRSSVLHST
QDLFLPFFSNVTWFHAISGTNGTKRFDNPVLPFNDGVYFASTEKSNIIRGWIFGTTL
DSKTQSLLIVNNATNVVIKVCEFQFCNDPFLGVYHKNNKSWMESEFRVYSSANNCT
FEYVSQPFLMDLEGKQGNFKNLREFVFKNIDGYFKIYSKHTPINLVRDLPQGFSALE
PLVDLPIGINITRFQTLLALHRSYLTPGDSSSGWTAGAAAYYVGYLQPRTFLLKYNEN
GTITDAVDCALDPLSETKCTLKSFTVEKGIYQTSNFRVQPTESIVRFPNITNLCPFGE
VFNATRFASVYAWNRKRISNCVADYSVLYNSASFSTFKCYGVSPTKLNDLCFTNVYA
DSFVIRGDEVRQIAPGQTGKIADYNYKLPDDFTGCVIAWNSNNLDSKVGGNYNYLY
RLFRKSNLKPFERDISTEIYQAGSTPCNGVEGFNCYFPLQSYGFQPTYGVGYQPYR
WVVLSFELLHAPATVCGPKKSTNLVKNKCVNFNFNGLTGTGVLTESNKKFLPFQQFG
RDIDDTTDAVRDPQTLEILDITPCSFGGVSVITPGTNTSNQVAVLYQGVNCTEVPVAI
HADQLTPTWRVYSTGSNVFQTRAGCLIGAEHVNNSYECDIPIGAGICASYQTQTNS
PGSASSVASQSIIAYTMSLGAENSVAYSNNSIAIPINFTISVTTEILPVSMTKTSVDCTM
YICGDSTECSNLLLQYGSFCTQLNRALTGIAVEQDKNTQEVFAQVKQIYKTPPIKDFG
GFNFSQILPDPSKPSKRSPIEDLLFNKVTLADAGFIKQYGDCLGDIAARDLICAQKFN
GLTVLPPLLTDEMIAQYTSALLAGTITSGWTFGAGPALQIPFPMQMAYRENGIGVTQ
NVLYENQKLIANQFNSAIGKIQDSLSSTPSALGKLQDVVNQNAQALNTLVKQLSSNF
GAISSVLNDILARLDPPEAEVQIDRLITGRLQSLQTYVTQQLIRAAEIRASANLAATKM
SECVLGQSKRVDFCGKGYHLMSFPQSAPHGVVFLHVTYVPAQEKNFTTAPAICHD
GKAHFPREGVFVSNGTHWFVTQRNFYEPQIITTHNTFVSGNCDVVIGIVNNTVYDPL
QPELDSFKEELDKYFKNHTSPDVDLGDISGINASVVNIQKEIDRLNEVAKNLNESLID
LQELGKYEQYIKWPGYIPEAPRDGQAYVRKDGEWVLLSTFLENLYFQGHHHHHH*
Candidate 2 (SEQ ID NO: 2): U4579IC070-7_S full(6P) R/x2-Fd
MGWSCIILFLVATATGVHSQCVNLTTRTQLPPAYTNSFTRGVYYPDKVFRSSVLHST
QDLFLPFFSNVTWFHAISGTNGTKRFDNPVLPFNDGVYFASTEKSNIIRGWIFGTTL
DSKTQSLLIVNNATNVVIKVCEFQFCNDPFLGVYHKNNKSWMESEFRVYSSANNCT
FEYVSQPFLMDLEGKQGNFKNLREFVFKNIDGYFKIYSKHTPINLVRDLPQGFSALE
PLVDLPIGINITRFQTLLALHRSYLTPGDSSSGWTAGAAAYYVGYLQPRTFLLKYNEN
GTITDAVDCALDPLSETKCTLKSFTVEKGIYQTSNFRVQPTESIVRFPNITNLCPFGE
VFNATRFASVYAWNRKRISNCVADYSVLYNSASFSTFKCYGVSPTKLNDLCFTNVYA
DSFVIRGDEVRQIAPCQTGKIADYNYKLPDDFTGCVIAWNSNNLDSKVGGNYNYLY
RLFRKSNLKPFERDISTEIYQAGSTPCNGVEGFNCYFPLQSYGFQPTYGVGYQPYR
WVVLSFELLHAPATVCGPKKSTNLVKNKCVNFNFNGLTGTGVLTESNKKFLPFQQFG
RDIDDTTDAVRDPQTLEILDITPCSFGGVSVITPGTNTSNQVAVLYQGVNCTEVPVAI
HADQLTPTWRVYSTGSNVFQTRAGCLIGAEHVNNSYECDIPIGAGICASYQTQTNS
RSVASQSIIAYTMSLGAENSVAYSNNSIAIPINFTISVTTEILPVSMTKTSVDCTMYICG
DSTECSNLLLQYGSFCTQLNRALTGIAVEQDKNTQEVFAQVKQIYKTPPIKDFGGFN
FSQILPDPSKPSKRSPIEDLLFNKVTLADAGFIKQYGDCLGDIAARDLICAQKFNGLT
VLPPLLTDEMIAQYTSALLAGTITSGWTFGAGPALQIPFPMQMAYRFNGIGVTQNVL
YENQKLIANQFNSAIGKIQDSLSSTPSALGKLQDVVNQNAQALNTLVKQLSSNFGAI
SSVLNDILARLDKCEAEVQIDRLITGRLQSLQTYVTQQLIRAAEIRASANLAATKMSE
CVLGQSKRVDFCGKGYHLMSFPQSAPHGVVFLHVTYVPAQEKNFTTAPAICHDGK
AHFPREGVFVSNGTHWFVTQRNFYEPQIITTHNTFVSGNCDVVIGIVNNTVYDPLQP
ELDSFKEELDKYFKNHTSPDVDLGDISGINASVVNIQKEIDRLNEVAKNLNESLIDLQ
ELGKYEQYIKWPGYIPEAPRDGQAYVRKDGEWVLLSTFLENLYFQGHHHHHH*
Candidate 3 (SEQ ID NO: 3): U2267IC070-5_NTD-RBD-Fd
MGWSCIILFLVATATGVHSQCVNLTTRTQLPPAYTNSFTRGVYYPDKVFRSSVLHST
QDLFLPFFSNVTWFHAISGTNGTKRFDNPVLPFNDGVYFASTEKSNIIRGWIFGTTL
DSKTQSLLIVNNATNVVIKVCEFQFCNDPFLGVYHKNNKSWMESEFRVYSSANNCT
FEYVSQPFLMDLEGKQGNFKNLREFVFKNIDGYFKIYSKHTPINLVRDLPQGFSALE
PLVDLPIGINITRFQTLLALHRSYLTPGDSSSGWTAGAAAYYVGYLQPRTFLLKYNEN
GTITDAVDCALDPLSETKCTLKSFTVEKGIYQTSNFRVQPTESIVRFPNITNLCPFGE
VFNATRFASVYAWNRKRISNCVADYSVLYNSASFSTFKCYGVSPTKLNDLCFTNVYA
DSFVIRGDEVRQIAPGQTGKIADYNYKLPDDFTGCVIAWNSNNLDSKVGGNYNYLY
RLFRKSNLKPFERDISTEIYQAGSTPCNGVEGENCYFPLQSYGFQPTYGVGYQPYR
WVVLSFELLHAPATVCGPKKSTNLVKNKCVNFGGSGGGYIPEAPRDGQAYVRKDGE
WVLLSTFLGGGGSHHHHHH*

1. A recombinant protein derived from the spike protein of severe acute respiratory syndrome coronavirus 2 (SARS-CoV-2), wherein the recombinant protein consists of an amino acid sequence selected from the group consisting of SEQ ID NO: 1, SEQ ID NO: 2, and SEQ ID NO: 3.

2. (canceled)

3. (canceled)

4. A vaccine composition comprising the recombinant protein according to claim 1.

5. The vaccine composition according to claim 4, wherein the vaccine composition further comprises one or more components selected from the group consisting of preservatives, diluents, adjuvants and carriers.

6. The vaccine composition according to claim 5, wherein the adjuvants are alum.

7. The vaccine composition according to claim 4, wherein the vaccine composition is for the prevention of severe acute respiratory syndrome coronavirus type 2 (SARS-CoV-2) infection.

8. The vaccine composition according to claim 7, wherein the severe acute respiratory syndrome coronavirus 2 (SARS-CoV-2) infection disease is COVID-19.

9. The vaccine composition according to claim 7, wherein the severe acute respiratory syndrome coronavirus 2 (SARS-CoV-2) is at least one variant selected from the group consisting of alpha, beta, gamma, delta and omicron.

10. The vaccine composition according to claim 4, wherein the vaccine composition is used as one or more selected from the group consisting of a priming vaccine composition and a boosting vaccine composition.

11. A method for preventing severe acute respiratory syndrome coronavirus 2 (SARS-CoV-2) infection in a non-human mammal, comprising a step of administering the vaccine composition of claim 4.

12. The method for preventing severe acute respiratory syndrome coronavirus 2 (SARS-CoV-2) infection according to claim 11, wherein the step of administering comprises a step of inducing a priming immune response and a step of inducing a boosting immune response.


SEQUENCE LISTING (3 sequences)

SEQ ID NO: 1 - protein, 1256 aa, synthetic construct
MGWSCIILFLVATATGVHSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILARLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPGYIPEAPRDGQAYVRKDGEWVLLSTFLENLYFQGHHHHHH

SEQ ID NO: 2 - protein, 1252 aa, synthetic construct
MGWSCIILFLVATATGVHSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPCQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILARLDKCEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPGYIPEAPRDGQAYVRKDGEWVLLSTFLENLYFQGHHHHHH

SEQ ID NO: 3 - protein, 587 aa, synthetic construct
MGWSCIILFLVATATGVHSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFGGSGGGYIPEAPRDGQAYVRKDGEWVLLSTFLGGGGSHHHHHH